(19) United States
(12) Patent Application Publication
(10) Pub. No.: US 2007/0141017 A1
(43) Pub. Date: Jun. 21, 2007

(54) Penetration enhancing agent and method of its production from the hemp seeds

(75) Inventor: Irena Horska, Liberec (CZ)

Correspondence Address: HERSHKOVITZ & ASSOCIATES, 2845 DUKE STREET, ALEXANDRIA, VA, 22314, US

(73) Assignee: PARENTERAL, A.S., 500 11 HRADEC KRALOVE (CZ)

(21) Appl. No.: 10/582,979

(22) PCT Filed: Dec. 15, 2004
(86) PCT No.: PCT/CZ04/00086

§ 371 Date: Sep. 26, 2006

(30) Foreign Application Priority Data
Dec. 15, 2003 (CZ) PV 2003-3412

(51) Int. Cl. A61K 35/64; A61K 36/18; A61K 8/97

(52) U.S. Cl. 424/74; 424/776; 424/539

(57) ABSTRACT

The conveying and absorption agent, preferably for the active substances of the pharmaceutical, dietetic, and cosmetic preparations, consists of the extract from the hemp seeds, obtained by action of carbon dioxide. Advantageously, the extract is a component of the fat or wax base of the pharmaceutical or cosmetic preparation, preferably composed of the propolis relieved of its pollen contents. According to the method of production of the conveying and absorption agent from the hemp seeds for the active substances of the pharmaceutical, dietetic, and cosmetic preparations, the hemp seeds are ground down to hemp flour, being then pressure-extracted with carbon dioxide to the hemp oil. The hemp seeds are ground down to fine-grain flour, which is then powered into the extraction cartridges that are inserted into the extractor. Extractor is then closed and the carbon dioxide is then driven into it at the temperature between 35° C.-45° C. and the pressure between 25 MPa and 35 MPa, advantageously 40° C. and pressure 29 MPa. With the hemp oil extraction process slowed down, the carbon dioxide pressure in the extractor is reduced down to the ambient atmospheric pressure value and the hemp oil is separated from the carbon dioxide which is subsequently led away from the extractor to the reserve tank, where it will be stored in its supercritical condition. For removal of chlorophyll and waxes, 2%-35% in weight of the crushed silicon sand is mixed into the hemp oil extracted. Following the surface absorption, the crushed silicon sand is filtered out.

### FIELD OF TECHNOLOGY

The invention pertains to a conveying and absorption agent, preferably for the active substances of the pharmaceutical, dietetic, and cosmetic preparations. The invention further relates to a method of production of the conveying and absorption agent from the hemp seeds.

### BACKGROUND OF THE INVENTION

The pharmaceutical, dietetic, and cosmetic preparations rely on crude-oil, organic, or vegetable basis. The preparations that rest on the crude oil base, made up e.g. by a Vaseline or paraffin have a limited capacity to penetrate into the skin deeper layer. In order to enhance the capacity to penetrate into the deeper skin layers the organic-chemical conveyors or keratolythics and emulsifiers of a synthetic, unnatural origin are therefore preferably being added in particular into the ointment and cream bases. The ointment and cream bases originating in crude oil are not much able to bind upon themselves the active substances. The synthetic absorbents that are, however, unable to bind upon themselves the modern bioactive substances such as cytokines, CD monoclonal antibodies, derivates of arachid acid, low-molecular peptides, modern antibiotics, etc. are therefore used to convey the active substances into the deeper skin layers. Further disadvantage of the ointment and cream bases, having originated in crude oil, is the fact that they have no therapeutic effect and that the synthetic organic-chemical conveyors, emulsifiers, and conserving substances tend to evoke allergic and other dermal reactions. The ointment and cream bases of animal or vegetal origin used until now contain the aliphatic acids and fats that are relatively unstable, tend to oxidize, and—as such—presume the use of the synthetic organic-chemical stabilizers with the relatively high levels of concentration, evoking adverse dermal responses. Although the ointment and cream bases of animal or vegetal origin are also capable to penetrate into the deeper skin layers, they have, however, a very limited capacity to bind upon themselves the bioactive substances such as the cytkins, CD monoclonal antibodies, arachid acid derivates low-molecular peptides, modern antibiotics, etc., and cannot therefore convey efficiently the active substances into the deeper skin layers. The ointment and cream bases of an animal origin, fats, lard, sheep wool fats, sperm whale stuff, and bee wax, as well as the ointment and cream bases of vegetal origin, helianthi oleum, arachidis oleum types, lino oleum, amygdalae oleum, ricini oleum oil types, herbal oils are being combined, preferably by mixing them together, but these combined bases neither remove the necessity to use the, organic-chemical conveyors, emulsifiers, and conserving substances of a synthetic, non-natural origin nor have any better capacity to bind upon themselves and convey the bioactive substances into the deeper skin layers.

A dermal inflammation healing agent, using the oil, cold-pressed out of the hemp seed, as an active substance is known from the specification DE 10041700. This dermal inflammation healing agent can, in accordance with the specification DE 10041700, be used in an emulsion with emulsifier such as Lizitin, for application onto the inflamed portions of skin. But the inflammation healing agent as per Volume DE 10041700 does not have any satisfactory effect and its applicability period/shelf life is short. When used, the preparation presumes a simultaneous application of the adversely acting synthetic stabilizers and emulsifiers. A cosmetic and therapeutic agent, composed of the hemp oil which is relieved of the oxidation-prone additives that are degrading this non-refined hemp oil via a bacterial biodegradation and that are evoking both the chemical and pharmacological instability, is known from the Volume CZ—U1-13335. When improved like this, the hemp oil can better counteract the action of the UV radiation and has an enhanced capacity to bind upon itself other pharmaceutics. Production of the cosmetic and therapeutic agent per Volume CZ—U1-13335 consists of multiple stages, being very demanding in technological terms, and its manufacturing cycle is very long. Removal of the oxidation-prone additives is incomplete.

Object of the invention is to avoid the disadvantages of the so far known bases of the pharmaceutical, dietetic, and cosmetic preparations and to provide a natural, more efficient active substance conveyor and absorbent, possessing moreover a better capacity to bind upon itself the bioactive substances, to penetrate into the deeper skin layers, and to convey the bioactive substances without the use of any synthetic carriers, featuring a longer utility period without the necessity to use any conservation chemicals, which—in its primary composition—would also show—without any additives—healing and relaxing effects, preferably in case of the dermal diseases and also contain—in its primary composition—a natural UV protective factor. Furthermore, the objective of the invention is to provide the method of production of the natural conveying and absorption agent based on the hemp oil in accordance with which it would be possible to produce this agent with these properties easily and reliably in the industrial conditions.

### SUBJECT-MATTER OF THE INVENTION

To a major extent the disadvantages of the current state of technology are eliminated and the invention objectives achieved by a conveying and absorption agent, preferably for the active pharmaceutical, dietetic and cosmetic substances, consisting of the matter extracted from the hemp seeds by carbon dioxide. Advantageously, the extract is a component of a fat, Vaseline or wax base of the pharmaceutical or cosmetic preparation. Advantageously, the extract is a component of the pharmaceutical or cosmetic preparation's base made up of propolis disposed of its pollen fraction. Advantageously, the extract weight share in the fat, wax, Vaseline, or propolis base or in the mixture of these bases equals to at least 0.5% in weight. Advantageously, one weight share of the extract creates a mixture with two weight shares of the solution containing up to 30% in weight of sodium bicarbonate. According to the method of production of the conveying and absorption agent from the hemp seeds for the active substances of the pharmaceutical, dietetic, and cosmetic preparations, the hemp seeds are milled down to hemp flour, then pressure-extracted by means of the carbon dioxide to hemp oil. Hemp seeds are milled down to a fine hemp flour, the milled down hemp flour is then poured into the extraction cartridges that are then inserted into the extractor; the extractor gets closed and carbon dioxide is driven into it at the temperature between about 35° C.-45° C. and under pressure between 25 MPa-35 MPa, advantageously at 40° C. and the pressure 20 MPa; with the hemp oil extraction process slowed down the carbon dioxide pressure in the extractor is reduced down to the value of the ambient atmospheric pressure and the hemp oil is separated from the carbon dioxide, then the carbon dioxide is taken out of the extractor to a reserve tank and stored there in its supercritical condition. In order to remove chlorophyll and waxes, 2%-35% in weight of the crushed silicon sand is mixed into the extracted hemp oil; following the surface absorption, the crushed silicon sand is filtered out.

The cosmetic, dietetic, and therapeutic preparations containing a conveying and absorption agent for conveying the active substances according to the invention have better efficiency as the conveying and absorption agent has a strong capacity to bind upon itself the bioactive substances. Unlike the agents known until now, the conveying and absorption agent according to the invention is able to bind upon itself a broader spectrum of the bioactive substances such as e.g. cytokines, CD monoclonal antibodies, arachid acid derivates, low-molecular peptides, modern antibiotics, and other substances, to penetrate and convey them into the deeper skin layers without the use of any synthetic carriers. The conveying and absorption agent according to the invention does not irritate skin, but on the contrary has more bio-chemical qualities, is healing eczema, acne, and relaxes notably psoriasis without application of any other active substances e.g. tar, hormonal substantia, contains a natural UV filter and a minimum of the substances capable of oxidation or reduction. Cosmetic and therapeutic preparations that contain the conveying and absorption agent according to the invention are stable for the period of at least two years without the necessity to apply any chemical conserving substances. The cosmetic and therapeutic preparation that contains the conveying and absorption agent according to the invention features a high concentration of linoleic acid, linolenic acid, gamma-linolenic acid, prostaglandins, prostacyclines, tromboxanes, leucotriens, and high level of purity, as it almost fails to contain the substances from the group of proteins, amino acids, and sugars. Major advantage rests in the fact, that when combined with the crude oil, animal, and other vegetal bases, the conveying and absorption agent according to the invention does not lose its efficiency and retains all the above-mentioned characteristics. An advantage of the method of production of the conveying and absorption agent according to the invention and the cosmetic and therapeutic preparations that contain the conveying and absorption agent according to the invention rests in its efficiency given by the fact that the above-mentioned bioactive substances that—thanks to their effects—influence the cellular nutrition in the skin bottom layers, making thus the therapy more effective, are remaining in the hemp oil. The conveying and absorption agent according to the invention also manifested itself as having positive impacts on the human health as a food ingredient as well.

### EXAMPLES OF EXECUTION

The following examples demonstrate usage of the hemp oil based conveying and absorption agent according to the invention for production of the ointment and cream bases.

### Example 1

Cosmetic and therapeutic preparation based on the hemp oil and consisting of 0.5% in weight of the hemp oil extracted from the hemp seeds by means of carbon dioxide, 84.5% in weight of the Flava Vaseline, and 15% in weight of the propolis relieved of its pollen share.

### Example 2

Cosmetic and therapeutic preparation based on the hemp oil and consisting of 5% in weight of the hemp oil extracted from the hemp seeds by means of carbon dioxide, 80.0% in weight of the lard, and 15% in weight of propolis relieved of its pollen share.

### Example 3

Cosmetic and therapeutic preparation based on the hemp oil and consisting of 20% in weight of the hemp oil extracted from the hemp seeds by means of carbon dioxide, 50% in weight of hard paraffin, and 30% in weight of propolis relieved of its pollen share.

### Example 4

Cosmetic and therapeutic preparation based on the hemp oil and consisting of 50% in weight of the hemp oil extracted from the hemp seeds by means of carbon dioxide, 40% in weight of yellow wax, and 10% in weight of ricini oleum.

### Example 5

Cosmetic and therapeutic preparation based on the hemp oil and consisting of 70% in weight of the hemp oil extracted from the hemp seeds by means of carbon dioxide, 20% in weight of white wax, 5% in weight of arachidis oleum, and 5% in weight of propolis relieved of its pollen share.

### Example 6

Therapeutic preparation based on the hemp oil and consisting of 95% in weight of the hemp oil extracted from the hemp seeds by means of carbon dioxide, 4% in weight of the sheep wool, and 1% in weight of propolis relieved of its pollen share.

### Example 7

Therapeutic preparation based on the hemp oil and consisting of 99.5% in weight of the hemp oil extracted from the hemp seeds by means of carbon dioxide, and 0.5% in weight of propolis relieved of its pollen share.

### Example

### 8

Therapeutic and dietetic preparation in the form of capsules as a food ingredient based on the hemp oil and consisting of 100% in weight of the hemp oil extracted from the hemp seeds by means of carbon dioxide.

### Example 9

Therapeutic preparation based on the hemp oil and consisting of 33.33% in weight of the hemp oil extracted from the hemp seeds by means of carbon dioxide, 40% in weight of amygdalea oleum, 20% in weight of sodium bicarbonate, and 6.66% in weight of propolis relieved of its pollen share.

### Example 10

Cosmetic and therapeutic preparation based on the hemp oil and consisting of the mixture of 33.33% in weight of the hemp oil extracted from the hemp seeds by means of carbon dioxide, 50% in weight of helianthi oleum and 10% in weight of sodium bicarbonate, and 6.66% in weight of propolis relieved of its pollen share.

According to the method of production of the conveying and absorption agent according to the invention, the hemp seeds are ground down to fine-grain flour which is then poured into the extraction cartridges that are then inserted into the extractor. The extractor is filled with the extraction cartridges with hemp flour and closed. Then, carbon dioxide is driven into the extractor at the temperature of 30° C.-45° C. and pressure of 25 MPa-35 MPa, advantageously at 40° C. and pressure 29 MPa. Within the scope of the above temperature and pressure values, the method of production is highly efficient, but this efficiency tends to drop sizably beyond this scope. By virtue of the carbon dioxide pressure the hemp oil gets extracted from the hemp flour. With the hemp oil extraction slowed down the carbon dioxide pressure in the extractor falls down to the value of the ambient atmospheric pressure and the hemp oil gets separated from the carbon dioxide. Then, the carbon dioxide is fully drawn out of the extractor and led into the reserve tank where it is then being stored in its supercritical condition. During the extraction, the carbon dioxide action upon the hemp flour is quite inert, not causing any chemical reactions. No reactions take place during the extraction; no new chemical bonds arise in the components involved in the extraction. Carbon dioxide is inert to proteins, sugars, and starches. In comparison with method of hemp oil production by cold pressing of the hemp seeds, there is no need to refine the hemp oil when extracted from the ground hemp seeds by means of the carbon dioxide or to subject it to any other modifications. Preferably the gamma-linolenic acid and arachid acid, as well as other substances will remain well preserved in the hemp oil extracted. Where a more transparent, lighter-shaded oil has to be gained and where the chlorophyll and waxes have to be removed, the filtration can advantageously be carried out by means of an inert diacomateous earth—a special sand, which (making use of the physical processes) binds upon itself these unwanted substances. Within this filtration technique, the diacomateous earth is mixed into the hemp oil in the ratio of 2%-35% in weight, being then filtered, advantageously through a multi-cloth filter in which, in order to get the active surface as large as possible, a set of cloth-coated frames, arresting the chlorophyll and wax wrapped diacomateous earth while the oil is poured through, is embedded. The ratio from 2%-35% in weight of the crushed silicon sand in the hemp oil appeared advantageous in respect of the efficient absorption of the unwanted substances on the surfaces of the crushed silicon sand. Advantageously, this proportion can be narrowed to 5%-20% in weight, when a spontaneous surface absorption takes place. When obtained by extraction, the hemp oil is showing the following typical parameters:
density 0.910-0.926 g/cm3
refraction index 1.472-1.480
peroxide number ≦15 O2/kg
number of acidity >25.0 mg KOH/g
iodine number 150-170 g as I2/100

When extracted with carbon dioxide, hemp oil contains in high concentration the substances from the group of linoleic acid, linolenic acid, gamma-linolenic acid, dihomogamalinolene acid, arachidine acide, prostaglandins, prostacyclines, tromboxanes, leucotriens and reaches a high level of purity, as it is quite disposed of the substances from the group of proteins, amino acids, and sugars.

### INDUSTRIAL USE

According to the invention, the conveying and absorption agent can be used as a part of the cosmetic, pharmaceutical, and dietetic preparation's bases, preferably as a part of the bases of the ointment and cream preparations being applied in order to relax, cure eczema, acne, and for substantial relaxing of psoriasis, as a drug and as a part of an ointment base for the subsequent mixing in of various substances, extracts, bioactive substances, pharmaceutically recognized substances, and as a component in the cosmetics, preferably in the suntan creams and skin regenerating creams, as well as in production of all other skin-friendly cosmetic preparations, such as soap, shampoo, gel, pharmaceutical and dietetic preparations.

1. Hemp seed oil for transfer and absorption of active substances of pharmaceutical and cosmetic preparations into the skin, characterised by that
it is a product of extraction of the milled down hemp seeds by means of carbon dioxide.

2. Hemp seed oil according to the claim 1, characterised by that
one weight share of the extract creates a mixture with two weight shares of the solution containing up to 30% in weight of sodium bicarbonate.

3. A method of production of hemp seed oil for transfer and absorption of active substances of pharmaceutical and cosmetic preparations into the skin, characterised by that
the hemp seeds are milled down to hemp flour, then pressure-extracted by means of the carbon dioxide to hemp oil.

4. A method of production according to the claim 3, characterised by that
hemp seeds are milled down to a fine hemp flour, the milled down hemp flour is then poured into the extraction cartridges that are then inserted into the extractor; the extractor gets closed-and carbon dioxide is driven into it at the temperature between about 35° C.-45° C. and under pressure between 25 MPa-35 MPa, advantageously at 40° C. and the pressure 20 MPa, with the hemp oil extraction process slowed down the carbon dioxide pressure in the extractor is reduced down to the value of the ambient atmospheric pressure and the hemp oil is separated from the carbon dioxide, then the carbon dioxide is taken out of the extractor to a reserve tank and stored there in its supercritical condition.

5. A method of production according to claim 3, wherein
2%-35% in weight of the crushed silicon sand is mixed into the extracted hemp oil for removal of the chiorophyll and waxes; following the surface absorption, the crushed silicon sand is filtered out.

6. A use of the hemp seed oil manufactured by a method according to claim 3 for transfer and absorption of active substances of pharmaceutical and cosmetic preparations into the skin.

7. A method of production according to claim 4, wherein
2%-35% in weight of the crushed silicon sand is mixed into the extracted hemp oil for removal of the chiorophyll and waxes; following the surface absorption, the crushed silicon sand is filtered out.

8. A use of the hemp seed oil manufactured by a method according to claim 4 for transfer and absorption of active substances of pharmaceutical and cosmetic preparations into the skin.

9. A use of the hemp seed oil manufactured by a method according to one of the claims 5 for transfer and absorption of active substances of pharmaceutical and cosmetic preparations into the skin.

10. A use of the hemp seed oil manufactured by a method according to claim 7 for transfer and absorption of active substances of pharmaceutical and cosmetic preparations into the skin.
